(19) United States
(12) Patent Application Publication
(10) Pub. No.: US 2013/0316454 A1
(43) Pub. Date: Nov. 28, 2013

(54) Methods for Producing Tissue Scaffold Directing Differentiation of Seeded Cells and Tissue Scaffolds Produced Thereby

(71) Applicants: Helen H. Lu, New York, NY (US); Shang-pin Kwei, Boston, MA (US); Natalie L. Leong, Los Angeles, CA (US); Marissa R. Solomon, New York, NY (US); Siddarth D. Subramony, Little Neck, NY (US)

(72) Inventors: Helen H. Lu, New York, NY (US); Shang-pin Kwei, Boston, MA (US); Natalie L. Leong, Los Angeles, CA (US); Marissa R. Solomon, New York, NY (US); Siddarth D. Subramony, Little Neck, NY (US)

(73) Assignee: The Trustees of Columbia University in the City of New York

(21) Appl. No.: 13/806,293

(22) PCT Filed: Jun. 22, 2011
(86) PCT No.: PCT/US11/41391

§ 371 Date: Aug. 5, 2013

Related U.S. Application Data
(60) Provisional application No. 61/398,265, filed on Jun. 22, 2010.
(60) Provisional application No. 61/519,461, filed on May 23, 2011.
(60) Provisional application No. 61/519,460, filed on May 23, 2011.
(60) Provisional application No. 61/519,491, filed on May 23, 2011.

(51) Int. Cl. C12N 5/077

(52) U.S. Cl. CPC C12N 5/0656; C12N 5/0654; C12N 5/0655
USPC 435/377

(57) ABSTRACT

Methods for producing a tissue scaffold which direct differentiation of seeded stem cells on the scaffold to a selected cell type and tissue scaffolds produced thereby are provided.

This patent application claims the benefit of priority from U.S. Provisional Application Ser. No. 61/398,265, filed Jun. 22, 2010, U.S. Provisional Application Ser. No. 61/519,491, filed May 23, 2011, U.S. Provisional Application Ser. No. 61/519,461, filed May 23, 2011, and U.S. Provisional Application Ser. No. 61/519,460, filed May 23, 2011, teachings of each of which are herein incorporated by reference in their entireties.

### STATEMENT REGARDING FEDERALLY SPONSORED RESEARCH OR DEVELOPMENT

This invention was made with government support under NSF Graduate Fellowship (GK-12) awarded by the National Science Foundation and Grant Numbers AR 055280-02, AR 052402 and AR 056459-02 awarded by the National Institutes of Health—National Institute of Arthritis and Musculoskeletal and Skin Diseases (NIH-NIAMS). The government has certain rights in the invention.

### BACKGROUND

Injuries to connective tissues such as tendons or ligaments are a common clinical problem treated traditionally by allografts, xenografts and autografts, as well as prosthetic devices, due to the poor regeneration ability of tendons. However, these traditional treatments suffer from disadvantages including donor site morbidity and risk of disease transmission, as well as limited long-term functionality.

Articular cartilage is an avascular, aneural tissue which also has limited capacity for self-repair. Current strategies for cartilage repair such as microfracture, mosaicplasty, lavage and periosteal grafts result in sup-optimal clinical outcomes due to donor site morbidity, fibrous tissue formation and insufficient graft integration.

Bone is also one of the most commonly replaced organs of the body with over 275,000 of the 1-2 million fractures being treated each year in the United States requiring bone grafting (Delacure, M. D. Otalarnygol. Clin. North Am, 1994 27(5):859-74; Langer, R. and Vacanti, J. P. Science 1993 260(5110):920-6). Donor site morbidity and risk of disease transmission have also limited the use of allografts and xenografts in bone grafting. Autografts are also limited by supply as well as risk of tissue harvesting leading to donor site morbidity.

### SUMMARY

This application provides for direction of stem cell differentiation on a tissue scaffold through biomaterial design. By selecting biomaterial/scaffold design parameters including composition, bioactivity, biomimetic architecture, physical or mechanical stimulation and/or chemical stimulation, the inventors show herein directed differentiation of stem cells into osteoblasts, chondrocytes, fibrochondrocytes or fibroblasts.

Accordingly, an aspect of this application relates to a method for producing a tissue scaffold which directs differentiation of seeded stem cells on the scaffold to a selected cell type. In this method, a substrate for the tissue scaffold which directs differentiation of stem cells seeded on the tissue scaffold to the selected cell type is selected. An architecture for the tissue scaffold which directs differentiation of stem cells seeded on the tissue scaffold to the selected cell type is also selected. A tissue scaffold with the selected architecture is then produced from the selected substrate and the tissue scaffold is seeded with stem cells so that they differentiate into the selected cells.

In one embodiment of this method, the tissue scaffold produced in accordance with this method is exposed to a physical or mechanical stimulation and/or a chemical stimulation which directs differentiation of the seeded stem cells on the scaffold to the selected cell type.

In one embodiment, the substrate, architecture and/or physical or mechanical stimulation and/or chemical stimulation are selected to direct seeded stem cells to differentiate on the tissue scaffold into osteoblasts.

In one embodiment, the substrate, architecture and/or physical or mechanical stimulation and/or chemical stimulation are selected to direct seeded stem cells to differentiate on the tissue scaffold into fibrochondrocytes.

In one embodiment, the substrate, architecture and/or physical or mechanical stimulation and/or chemical stimulation are selected to direct seeded stem cells to differentiate on the tissue scaffold into chondrocytes.

In one embodiment, the substrate, architecture and/or physical or mechanical stimulation and/or chemical stimulation are selected to direct seeded stem cells to differentiate on the tissue scaffold into fibroblasts.

Another aspect of this application relates to tissue scaffolds produced in accordance with this method of selecting a substrate, architecture and/or physical or mechanical stimulation and/or chemical stimulation which direct stem cells seeded on the tissue scaffold to differentiate into a selected cell type.

In one embodiment, the substrate, architecture and/or physical or mechanical stimulation and/or chemical stimulation of the tissue scaffold are selected so that stem cells seeded on the produced tissue scaffold are directed to differentiate into osteoblasts.

In one embodiment, the substrate, architecture and/or physical or mechanical stimulation and/or chemical stimulation of the tissue scaffold are selected so that stem cells seeded on the produced tissue scaffold are directed to differentiate into fibrochondrocytes.

In one embodiment, the substrate, architecture and/or physical or mechanical stimulation and/or chemical stimulation of the tissue scaffold are selected so that stem cells seeded on the produced tissue scaffold are directed to differentiate into chondrocytes.

In one embodiment, the substrate, architecture and/or physical or mechanical stimulation and/or chemical stimulation of the tissue scaffold are selected so that stem cells seeded on the produced tissue scaffold are directed to differentiate into fibroblasts.

### BRIEF DESCRIPTION OF THE FIGURES

FIG. 1 is a diagram depicting preparation of an osteogenic tissue scaffold embodiment produced by selecting biomaterial/scaffold design parameters in accordance with the method described herein.

FIG. 2 shows photomicrographs of cell distribution and viability of hMSCs seeded on an osteogenic tissue scaffold produced as depicted in FIG. 1 (PLGA-BG) compared to tissue scaffolds without the selected biomaterial/scaffold design parameters directing differentiation to osteoblasts (PLGA and PLGA-HA).

FIG. 3 is a bargraph comparing proliferation results of hMSCs seeded on an osteogenic tissue scaffold produced as depicted in FIG. 1 (PLGA-BG) compared to tissue scaffolds without the selected biomaterial/scaffold design parameters directing differentiation to osteoblasts (PLGA and HA).

FIG. 4 is a bargraph comparing proliferation results of hMSCs seeded on an osteogenic tissue scaffold produced as depicted in FIG. 1 (PLGA-BG) compared to tissue scaffolds without the selected biomaterial/scaffold design parameters directing differentiation to osteoblasts in the presence of a standard osteogenic media (PLGA and PLGA-HA).

FIG. 5 is a bargraph comparing ALP activity of hMSCs seeded on an osteogenic tissue scaffold produced as depicted in FIG. 1 (PLGA-BG) compared to tissue scaffolds without the selected biomaterial/scaffold design parameters directing differentiation to osteoblasts (PLGA and PLGA-HA).

FIG. 6 is a bargraph comparing ALP activity of hMSCs seeded on an osteogenic tissue scaffold produced as depicted in FIG. 1 (PLGA-BG) compared to tissue scaffold without the selected biomaterial/scaffold design parameters directing differentiation to osteoblasts in the presence of a standard osteogenic media (PLGA and PLGA-HA).

FIG. 7 is a gel comparing expression of osteoblastic markers osteopontin (OPN), osteonectin (ON) and osteocalcin (OCN) 7 days after seeding of hMSCs on an osteogenic tissue scaffold produced as depicted in FIG. 1 (PLGA-BG) compared to tissue scaffolds without the selected biomaterial/scaffold design parameters directing differentiation to osteoblasts (PLGA and PLGA-HA).

FIG. 8 is a gel comparing expression of osteoblastic markers osteopontin (OPN), osteonectin (ON) and osteocalcin (OCN) 7 days after seeding of hMSCs on an osteogenic tissue scaffold produced as depicted in FIG. 1 (PLGA-BG) compared to tissue scaffolds without the selected biomaterial/scaffold design parameters directing differentiation to osteoblasts in the presence of osteogenic media (PLGA and PLGA-HA).

FIG. 9 is a schematic of the experimental design also described in Example 2 of this application wherein osteogenic differentiation potential of media derived from an osteogenic tissue scaffold produced as depicted in FIG. 1 (BG) was compared to media derived from tissue scaffolds without the selected biomaterial/scaffold design parameters directing differentiation to osteoblasts (PLGA, HA and TCP).

FIGS. 10A and 10B are bargraphs comparing normalized ALP activity of hMSCs after culturing for 7, 14, 21 and 28 days in media derived from an osteogenic tissue scaffold (BG) as compared to media derived from tissue scaffolds without the selected biomaterial/scaffold design parameters directing differentiation to osteoblasts (PLGA, HA and TCP) as described in FIG. 9 in the absence (FIG. 10A) and the presence of standard osteogenic media (FIG. 10B).

FIG. 11 shows photomicrographs comparing cell viability and morphology of hMSCs and human rotator cuff fibroblasts (hRCFs) after culturing for 1 and 14 days on an embodiment of a fibrogenic tissue scaffold produced in accordance with the method described in this application in Example 3 (aligned) compared to a tissue scaffold without the selected biomaterial/scaffold design parameters directing differentiation to fibroblasts (unaligned).

FIGS. 12A and 12B are bargraphs comparing integrin αV expression of hRCFs (FIG. 12A) and hMSCs (FIG. 12B) after culturing for 1, 3 and 14 days on an embodiment of a fibrogenic tissue scaffold produced in accordance with the method described in this application in Example 3 (aligned) compared to a tissue scaffold without the selected biomaterial/scaffold design parameters directing differentiation to fibroblasts (unaligned). “*” represents significant difference between groups (p<0.05) as assessed by Tukey-HSD post-hoc test.

FIGS. 13A and 13B are bargraphs comparing integrin α5 expression of hRCFs (FIG. 13A) and hMSCs (FIG. 13B) after culturing for 1, 3 and 14 days on an embodiment of a fibrogenic tissue scaffold produced in accordance with the method described in this application in Example 3 (aligned) compared to a tissue scaffold without the selected biomaterial/scaffold design parameters directing differentiation to fibroblasts (unaligned). “*” represents significant difference between groups (p<0.05) as assessed by Tukey-HSD post-hoc test.

FIGS. 14A and 14B are bargraphs comparing integrin α2 expression of hRCFs (FIG. 14A) and hMSCs (FIG. 14B) after culturing for 1, 3 and 14 days on an embodiment of a fibrogenic tissue scaffold produced in accordance with the method described in this application in Example 3 (aligned) compared to a tissue scaffold without the selected biomaterial/scaffold design parameters directing differentiation to fibroblasts (unaligned). “*” represents significant difference between groups (p<0.05) as assessed by Tukey-HSD post-hoc test.

FIGS. 15A and 15B are bargraphs comparing integrin expression of hRCFs (FIG. 15A) and hMSCs (FIG. 15B) after culturing for 1, 3 and 14 days on an embodiment of a fibrogenic tissue scaffold produced in accordance with the method described in this application in Example 3 (aligned) compared to a tissue scaffold without the selected biomaterial/scaffold design parameters directing differentiation to fibroblasts (unaligned. “*” represents significant difference between groups (p<0.05) as assessed by Tukey-HSD post-hoc test.

FIG. 16 shows a mechanical stimulation device which directs differentiation of hMSCs to fibroblasts as described in this application in Example 4.

FIG. 17A is a bargraph quantifying cell proliferation and FIG. 17B is photomicrographs depicting cell proliferation of hMSCs after culturing for 1, 7, 14 and 28 days on an embodiment of a fibrogenic tissue scaffold produced in accordance with the method described in this application in Example 3 (unloaded) compared to a fibrogenic tissue scaffold produced in accordance with the method described in this application in Example 4 (loaded).

FIGS. 18A through C are photomicrographs (FIG. 18A) depicting cell alignment of hMSCs after culturing for 14 days on an embodiment of a fibrogenic tissue scaffold produced in accordance with the method described in this application in Example 3 (unloaded) compared to a fibrogenic tissue scaffold produced in accordance with the method described in this application in Example 4 (loaded) as well as a graph (FIG. 18B) and with tabular results (FIG. 18C) showing circular statistical analysis of the images using Fiber 3 software (Costa et al. Tissue Engineering 2003 9(4):567-77).

FIGS. 19A through C shows results of matrix production by hMSCs after culturing on an embodiment of a fibrogenic tissue scaffold produced in accordance with the method described in this application in Example 3 (unloaded) compared to a fibrogenic tissue scaffold produced in accordance with the method described in this application in Example 4 (loaded). Total collagen at day 14 and 28 (FIG. 19A) and day 1, 7, 14 and 28 (FIG. 19B) as well as collagen I and collagen III (FIG. 19C) were measured.

FIGS. 20A through D are bargraphs showing results of fibroblastic differentiation of hMSCs as determined by measuring collagen I (FIG. 20A), collagen III (FIG. 20B), fibronectin (FIG. 20C) and tenascinC (FIG. 20D) after culturing for 1, 7, 14 and 28 days on an embodiment of a fibrogenic tissue scaffold produced in accordance with the method described in this application in Example 3 (unloaded) compared to a fibrogenic tissue scaffold produced in accordance with the method described in this application in Example 4 (loaded). “*” represents significant difference between groups (p<0.05) as assessed by Tukey-HSD post-hoc test.

FIGS. 21A through D are bargraphs showing results of expression of integrin α2 (FIG. 21A), integrin αV (FIG. 21B), integrin (15 (FIG. 21C) and integrin β1 (FIG. 21D) by hMSCs after culturing for 1, 7, 14 and 28 days on an embodiment of a fibrogenic tissue scaffold produced in accordance with the method described in this application in Example 3 (unloaded) compared to a fibrogenic tissue scaffold produced in accordance with the method described in this application in Example 4 (loaded). “*” represents significant difference between groups (p<0.05) as assessed by Tukey-HSD post-hoc test.

FIGS. 22A through C are bargraphs showing mechanical properties include elastic modulus (FIG. 22A), ultimate stress (FIG. 22B) and yield stress (FIG. 22C) of hMSCs after culturing for 1, 7, 14 and 28 days on an embodiment of a fibrogenic tissue scaffold produced in accordance with the method described in this application in Example 3 (unloaded) compared to a fibrogenic tissue scaffold produced in accordance with the method described in this application in Example 4 (loaded). “*” represents significant difference between groups (p<0.05) as assessed by Tukey-HSD post-hoc test.

FIGS. 23A and B compare proliferation of hMSCs cultured on an embodiment of a chondrogenic tissue scaffold produced in accordance with the method described in this application in Example 5 (20-1 group) compared to a tissue scaffold without the selected biomaterial/scaffold design parameters directing differentiation to chondrocytes (20-0 group). FIG. 23A shows cells at Day 42 at 10× magnification, scale bar 200 μm (FIG. 23A). Cell proliferation data obtained with the PICOGREEN® ds DNA assay is depicted in FIG. 23B for days 1, 14, 28 and 42. Significant difference from day 1 is represented by *, from day 14 is represented by ̂, and from control is represented by **.

FIGS. 24A through E are bargraphs showing GAG production (FIG. 24A-B) and collagen production (FIG. 24C-D) normalized to wet weight and total cell number of hMSCs cultured on an embodiment of a chondrogenic tissue scaffold produced in accordance with the method described in this application in Example 5 (20-1 group) compared to a tissue scaffold without the selected biomaterial/scaffold design parameters directing differentiation to chondrocytes (20-0 group). Significant difference from day 1 is represented by *, from day 14 is represented by ̂, and from control is represented by **. FIG. 24E shows results from staining with Alcian Blue for sGAG for hMSCs cultured on an embodiment of a chondrogenic tissue scaffold (20-1 group) produced in accordance with the method described in this application in Example 5 compared to a tissue scaffold without the selected biomaterial/scaffold design parameters directing differentiation to chondrocytes (20-0 group) at magnification of 10× and a scale bar of 200 μm.

FIGS. 25A through E are bargraphs showing results from Real Time RT-PCR gene expression for hMSCs cultured on an embodiment of a chondrogenic tissue scaffold produced in accordance with the method described in this application in Example 5 (20-1 group) compared to a tissue scaffold without the selected biomaterial/scaffold design parameters directing differentiation to chondrocytes (20-0 group). Expression was measured for the following genes: SOX9 (FIG. 25A), aggrecan (FIG. 25B), collagen II (FIG. 25C), collagen I(FIG. 25D) and COMP (FIG. 25E) Genes were normalized to GAPDH and intensity was normalized to control (20-0 group) at day 14. Significant difference from day 14 is represented by *, from control is represented by **.

### DETAILED DESCRIPTION

### Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “aligned fibers” shall mean groups of fibers which are oriented along the same directional axis. Examples of aligned fibers include, but are not limited to, groups of parallel fibers.

As used herein, “ALP activity” shall mean alkaline phosphatase activity.

As used herein, a “biocompatible” material is a synthetic or natural material used to replace part of a living system or to function in intimate contact with living tissue. Biocompatible materials are intended to interface with biological systems to evaluate, treat, augment or replace any tissue, organ or function of the body. The biocompatible material has the ability to perform with an appropriate host response in a specific application and does not have toxic or injurious effects on biological systems. Nonlimiting examples of a biocompatible materials include a biocompatible ceramic, a biocompatible polymer or a biocompatible hydrogel.

As used herein, “biodegradable” means that the material, once implanted into a host, will begin to degrade.

As used herein, “biomimetic” shall mean a resemblance of a synthesized material to a substance that occurs naturally in a human body and which is not substantially rejected by (e.g., does not cause an unacceptable adverse reaction in) the human body. When used in connection with the tissue scaffolds, biomimetic means that the scaffold is substantially biologically inert (i.e., will not cause an unacceptable immune response/rejection) and is designed to resemble a structure (e.g., soft tissue anatomy) that occurs naturally in a mammalian, e.g., human, body and that promotes healing when implanted into the body.

As used herein, “chondrocyte” shall mean a differentiated cell responsible for secretion of extracellular matrix of cartilage.

As used herein, “chondrogenesis” shall mean the formation of cartilage tissue.

As used herein, “effective amount” shall mean a concentration, combination or ratio of one or more components added to the substrate which directs differentiation of stem cells to the selected cell type. Such components may include, but are not limited to, bioglass or glass ceramic, one or more extracellular matrix components, physical or mechanical stimulation and chemical stimulation such as media or growth factors which direct differentiation of stem cells to a selected cell type.

As used herein, “fibroblast” shall mean a cell which may be mesodermally derived that secretes proteins and molecular collagen including fibrillar procollagen, fibronectin and collagenase, from which an extracellular fibrillar matrix of connective tissue may be formed. Fibroblasts synthesize and maintain the extracellular matrix of many tissues, including but not limited to connective tissue. A “fibroblast-like cell” means a cell that shares certain characteristics with a fibroblast (such as expression of certain proteins).

As used herein, “fibrochondrocyte” shall mean a cell having features of chondrocytes and fibroblasts. Like chondrocytes, they have a rounded morphology and are protected by a territorial matrix. Like fibroblasts, the cells produce collagen-1, and like chondrocytes, these cells can produce collagen-2.

As used herein, “graft” shall mean the device to be implanted during medical grafting, which is a surgical procedure to transplant tissue without a blood supply, including but not limited to soft tissue graft, synthetic grafts, and the like.

As used herein, “hydrogel” shall mean any colloid in which the particles are in the external or dispersion phase and water is in the internal or dispersed phase.

As used herein, “microspheres”, mean microbeads, which are suitable, e.g., for cell attachment and adhesion. Microspheres of a tissue scaffold may be made from polymers such as aliphatic polyesters, poly(amino acids), copoly(ether-esters), polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, or biopolymers, or a blend of two or more of the preceding polymers. Preferably, the polymer comprises at least one of the following materials: poly(lactide-co-glycolide), poly(lactide) or poly(glycolide). More preferably, the polymer is poly(lactide-co-glycolide) (PLGA).

As used herein, “microfiber” shall mean a fiber with a diameter no more than 1000 micrometers.

As used herein, “nanofiber” shall mean a fiber with a diameter no more than 1000 nanometers.

In one embodiment, the microfibers and/or or nanofibers are comprised of a biodegradable polymer that is electrospun into a fiber. The microfibers and/or nanofibers of the scaffold are oriented in such a way (i.e., aligned or unaligned) so as to mimic the natural architecture of the soft tissue to be repaired. Moreover, the microfibers and/or nanofibers and the subsequently formed microfiber and/or nanofiber scaffold are controlled with respect to their physical properties, such as for example, fiber diameter, pore diameter, and porosity so that the mechanical properties of the microfibers and/or nanofibers and microfiber and/or nanofiber scaffold are similar to the native tissue to be repaired, augmented or replaced.

As used herein, “osteoblast” shall mean a bone-forming cell which may be derived from mesenchymal osteoprogenitor cells and which forms an osseous matrix in which it becomes enclosed as an osteocyte. The term may also be used broadly to encompass osteoblast-like, and related, cells, such as osteocytes and osteoclasts. An “osteoblast-like cell” means a cell that shares certain characteristics with an osteoblast (such as expression of certain proteins unique to bones), but is not an osteoblast. “Osteoblast-like cells” include preosteoblasts and osteoprogenitor cells.

As used herein, “osteogenesis” shall mean the production of bone tissue.

As used herein, “osteointegrative” means having the ability to chemically bond to bone.

As used herein, “polymer” means a chemical compound or mixture of compounds formed by polymerization and including repeating structural units. Polymers may be constructed in multiple forms and compositions or combinations of compositions.

As used herein, “porosity” means the ratio of the volume of interstices of a material to a volume of a mass of the material. As used herein, “porous” shall mean having an interconnected pore network.

As used herein, “soft tissue graft” shall mean a graft which is not synthetic, and can include autologous grafts, syngeneic grafts, allogeneic grafts, and xenogeneic graft. As used herein, “soft tissue” includes, as the context may dictate, tendon and ligament, as well as the bone to which such structures may be attached. Preferably, “soft tissue” refers to tendon- or ligament-bone insertion sites requiring surgical repair, such as for example tendon-to-bone fixation.

As used herein, “stem cell” means any unspecialized cell that has the potential to develop into many different cell types in the body, such as mesenchymal osteoprogenitor cells, osteoblasts, osteocytes, osteoclasts, chondrocytes, chondrocyte progenitor cells, fibrochondrocytes, fibroblasts and fibroblast progenitor cells. Nonlimiting examples of “stem cells” include mesenchymal stem cells, embryonic stem cells and induced pluripotent cells.

As used herein, “synthetic” shall mean that the material is not of a human or animal origin.

As used herein, all numerical ranges provided are intended to expressly include at least the endpoints and all numbers that fall between the endpoints of ranges.

The following embodiments are provided to further illustrate the methods of tissue scaffold production of this application. These embodiments are illustrative only and are not intended to limit the scope of this application in any way.

### Embodiments

Cells such as fibroblasts, when seeded on a tissue scaffold have a limited capacity to proliferate (Ge et al. Cell Transplant. 2005 14:573-83). However, inducing mesenchymal stem cells seeded on a tissue scaffold to differentiate into, for example, tendon-forming cells and avoiding ossification in vivo has been described as challenging, thus hindering their use (Yin et al. Biomaterials 2010 31:2163-2175; Harris et al. J. Orthop. Res. 2004 22:998-1003).

Provided in this disclosure are methods for producing tissue scaffolds which direct differentiation of seeded stem cells on the scaffold to a selected cell type. Also provided in this disclosure are tissue scaffolds produced by these methods.

The methods involve selecting a substrate from which the tissue scaffold is produced which will direct the stem cells seeded on the scaffold to differentiate to a selected cell type. The methods further involve selecting an architecture for the tissue scaffold which will direct the stem cells seeded on the scaffold to differentiate to the selected cell type. The tissue scaffold with the selected architecture is then produced from the selected substrate and seeded with stem cells so that they differentiate into the selected cell type. These methods may further comprise exposing the tissue scaffold to a physical or mechanical stimulation and/or a chemical stimulation which further enhances differentiation of stem cells seeded on the scaffold to the selected cell type. Preferred in this disclosure is that the selected cell type to which seeded stem cells are directed to differentiate be osteoblasts, chondrocytes, fibrochondrocytes or fibroblasts. Accordingly, in one embodiment, the tissue scaffolds produced in accordance with the methods disclosed herein are seeded with mesenchymal stem cells, also referred to herein as hMSCs, an unspecialized cell that has the potential to develop into many different cell types in the body, including, but not limited to, mesenchymal osteoprogenitor cells, osteoblasts, osteocytes, osteoclasts, chondrocytes, chondrocyte progenitor cells, fibrochondrocytes, fibroblasts and fibroblast progenitor cells. These adult bone marrow-derived stem cells (MSC) (Pittinger et al. Science 1999 284(5411):143-7; Caterson et al. Med Gen Med 2001 3(1):El; Altman et al. FASEB J 2002 162):270-2; Mao et al. Biol. Cell 2005 97(5):289-301) are used because they are physiologically relevant, well characterized, and can differentiate into osteoblasts, chondrocytes, fibrochondrocytes and fibroblasts. As will be understood by the skilled artisan upon reading this disclosure, alternative stem cells which can differentiate into osteoblasts, chondrocytes, fibrochondrocytes and fibroblasts can also be used.

In one embodiment, a method is provided for producing tissue scaffolds which direct differentiation of seeded stem cells on the scaffold to fibroblasts.

In this embodiment, the substrate selected comprises polymeric nanofiber and/or microfibers. Examples of polymers which can be selected for the substrate in this embodiment include, but are not limited to, biodegradable polymers selected from the group consisting of aliphatic polyesters, poly(amino acids), modified proteins, polydepsipeptides, copoly(ether-esters), polyurethanes, polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, modified polysaccharides, polycarbonates, polytyrosinecarbonates, polyorthocarbonates, poly(trimethylene carbonate), poly(phosphoester)s, polyglycolide, polylactides, polyhydroxybutyrates, polyhydroxyvalerates, polydioxanones, polyalkylene oxalates, polyalkylene succinates, poly(malic acid), poly(maleic anhydride), polyvinylalcohol, polyesteramides, polycyanoacrylates, polyfumarates, poly(ethylene glycol), polyoxaesters containing amine groups, poly(lactide-co-glycolides), poly(lactic acid)s, poly(glycolic acid)s, poly(dioxanone)s, poly(alkylene alkylate)s, biopolymers, collagen, silk, chitosan, alginate, and a blend of two or more of the preceding polymers. In one embodiment, the polymer comprises at least one of poly(lactide-co-glycolide), poly(lactide), and poly(glycolide). In one embodiment, the polymer is a copolymer, such as for example a poly(D,L-lactide-co-glycolide (PLGA). Advantages of a PLGA nanofiber and/or microfiber scaffold include that it is 1) biomimetic and guides tendon regeneration, 2) biodegradable and replaced by host tissue, 3) exhibit physiologically relevant mechanical properties, and 4) enable biological fixation of tendon-to-bone.

The ratio of polymers in the microfiber/nanofiber scaffold may be varied to achieve certain desired physical properties, including e.g., strength, ease of fabrication, degradability, and biocompatibility. In one embodiment, the ratio of polymers in the biocompatible polymer, e.g., the PLGA copolymer, is between about 25:75 to about 95:5. In one embodiment, the ratio of polymers in the biocompatible polymer, e.g., the PLGA copolymer, is between about 85:15. Generally, a ratio of about 25:75 in the PLGA copolymer will equate to a degradation time of about six months, a ratio of about 50:50 in the PLGA copolymer will equate to a degradation time of about twelve months, and a ratio of about 85:15 in the PLGA copolymer will equate to a degradation time of about eighteen months.

The architecture of the substrate selected for this fibrogenic tissue scaffold is aligned polymer microfibers and/or nanofibers.

In one embodiment of this method for production of a fibrogenic tissue scaffold, the produced tissue scaffold is exposed to physical or mechanical stimulation following seeding with the stem cells. Examples of physical or mechanical stimulation include, but are not limited to, cyclic tensile loading as described, for example, in PCT International Application No. PCT/US2009/06453, filed Dec. 8, 2009 providing configurable displacement and frequency application, and torsional loading as described, for example, by Altman et al. FASEB J 2002 16(2):270-2 and Moreau et al. Tiss Eng. A 2008 14(7):1161-72).

In another embodiment of this method for production of a fibrogenic tissue scaffold, chemical stimulation is applied to the microfiber and/or nanofiber scaffold. In one embodiment, the chemical stimulation comprises a growth factor. Examples of growth factors include, but are in no way limited to, cytokines such as bFGF and TGF-β.

In yet another embodiment of this method for production of a fibrogenic tissue scaffold, mechanical stimulation and chemical stimulation are applied to the microfiber and/or nanofiber scaffold.

A polymer nanofiber-based scaffold with aligned fibers was produced in accordance with the method of this disclosure and shown to guide adhesion of hMSCs and induce differentiation of hMSCs into fibroblast-like cells.

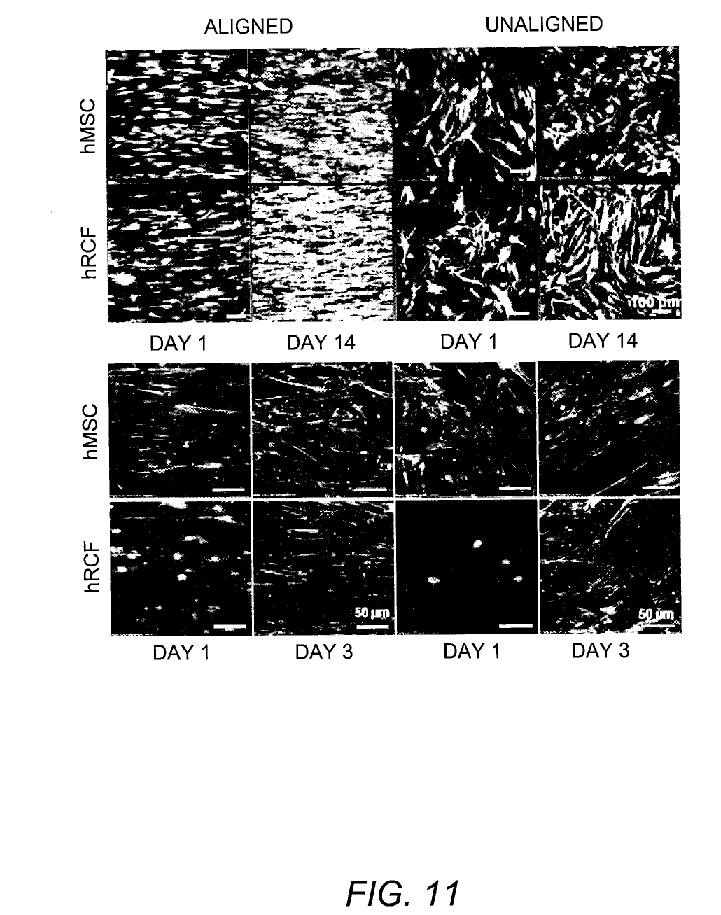

Cell viability and morphology of hMSCs and hRCFs seeded on the fibrogenic tissue scaffold produced in accordance with the method of this application is depicted in FIG. 11. It was found that both hMSCs and hRCFs were viable and grew over time. In addition, there was no apparent difference between the two cell types. Further, morphology was guided by nanofiber alignment. The hMSCs exhibited an elongated shape similar to fibroblasts on the aligned nanofiber scaffold produced in accordance with the method of this application. In contrast, the hMSCs exhibited an undesirable cuboidal appearance on the unaligned nanofiber tissue scaffold produced without the selected biomaterial/scaffold design parameters directing differentiation to fibroblasts.

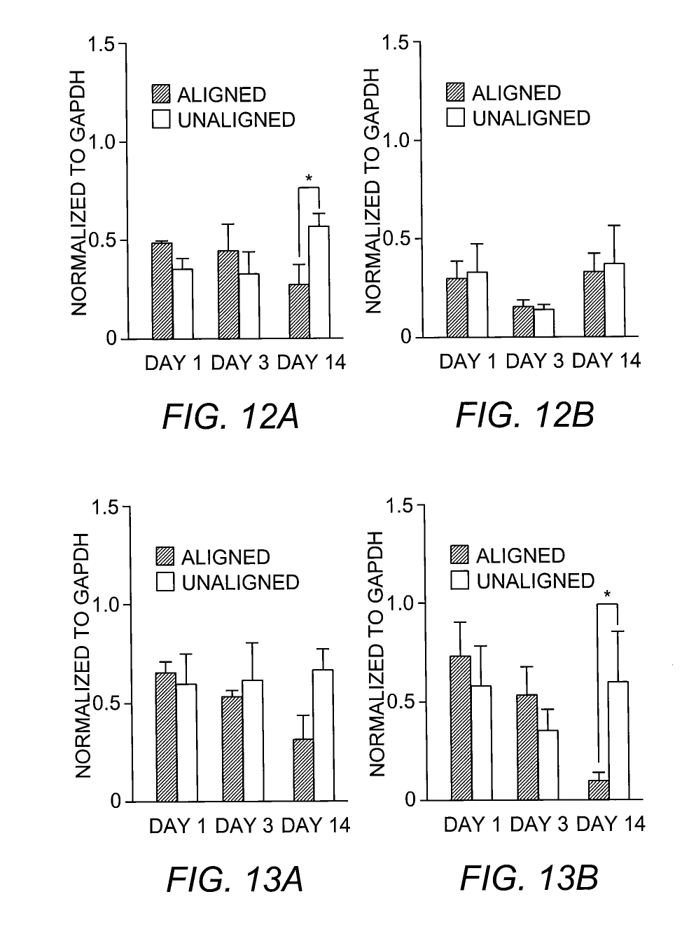

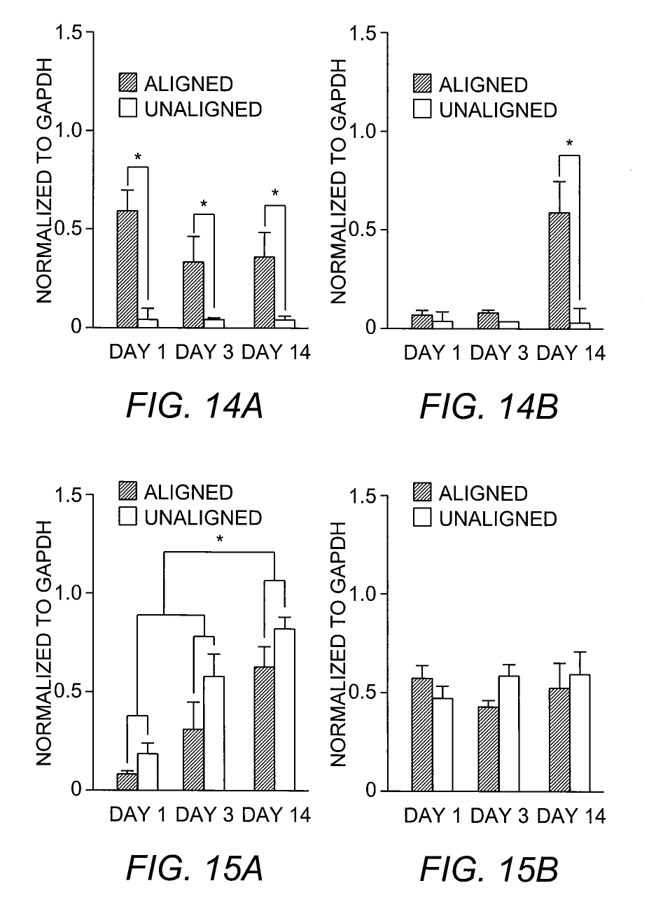

Integrin expression is important for cell matrix interactions, tendon and ligament healing and mechanotransduction (Singhvi et al. Biotechnol. Bioeng. 1994 43(8):764-71; Wang et al. J. Biomech. 2003 36(1):97-102; Harwood et al. Connect Tiss. Res. 1998 39(4):309-16; Banes et al. Biochem. Cell Biol. 1995 73(7-8):349-65; Schreck et al. J. Orthop. Res. 1995 13(2):174-83; Bhargava et al. J. Orthop. Res. 1999 17(5):748-54; Hannafin et al. 2006 J. Orthop. Res. 2006 24(2):149-58). Accordingly, the effect of nanofiber alignment on integrins α2, αV, α5, β1 was determined and is depicted graphically in FIGS. 12-15 and summarized in Table 1.

TABLE 1

| | hRCF | hMSC |
|---|---|---|
| Integrins | No significant | No significant |
| αV | differences observed | difference observed |
| | before day 14. | between groups. |
| | Expression of α2 | No significant |
| | increased significantly | difference observed |
| | on the unaligned | over time. |
| | scaffolds by day 14. | |
| Integrins | Expression of α5 | Significantly higher |
| α5 | comparable between | expression of α5 on |
| | substrates. | unaligned scaffold on |
| | Higher expression on | day 14 (6-fold |
| | aligned substrate on | increase). |
| | day 14. | Response mimicking that |
| | | of hRCF on unaligned by |
| | | day 14. |
| Integrins | Minimal expression of | Minimal expression of |
| α2 | α2 observed on | α2 observed initially |
| | unaligned group. | on either substrate. |
| | Significantly higher α2 | Aligned scaffolds up- |
| | expression in the | regulated α2 expression |
| | aligned group. | by day 14 (p < 0.05). |
| | | Response mimicking that |
| | | of hRCF on aligned by |
| | | day 14. |
| Integrins | Significantly higher β1 | No change in expression |
| β1 | expression on the | of β1 due to matrix |
| | aligned scaffold. | alignment. |
| | Difference between | Significant difference |
| | groups maintained over | observed between two |
| | time. | cell types. |

Thus, high α2 expression by both hRCFS and hMSCs was observed on the aligned nanofiber scaffolds produced in accordance with the method of this application. In contrast, the nanofiber tissue scaffold produced without the selected biomaterial/scaffold design parameters directing differentiation to fibroblasts induced higher αV expression in hRCF and elevated α5 in both hRCF and hMSC by day 14. Both αV and α5 have been associated with tendon and ligament healing (Harwood et al. Connect Tiss. Res. 1998 39(4):309-16, Schreck et al. J. Orthop Res. 1995 13(2):174-83). Thus, compared to the aligned nanofiber scaffold, a nanofiber tissue scaffold produced without the selected biomaterial/scaffold design parameters directing differentiation to fibroblasts may induce a scar healing response in hRCFs and hMSCs.

Overall these results confirm that the aligned nanofiber matrix produced in accordance with the method of this application elicits a more biomimetic response in hRCFs and directs the differentiation of hMSCs into hRCF-like cells. The cell-nanofiber interactions appear to be regulated by integrins.

In addition, these fibrogenic tissue scaffolds have mechanical properties comparable to those of the human ACL (Woo et al. J. Orthop. Rev. 1992 21(7):835-42) (Table 2)

TABLE 2

| | ACL | Nanofibers |
|---|---|---|
| Fiber Diameter | 45 | 615 ± 152 |
| (nm) | | |
| Elastic | 180 | 341 ± 30 |
| Modulus (MPa) | | |
| Yield Strength | 46.7 | 9.8 ± 1.1 |
| (MPa) | | |
| Ultimate | 75.8 | 12.0 ± 1.5 |
| Stress (MPa) | | |

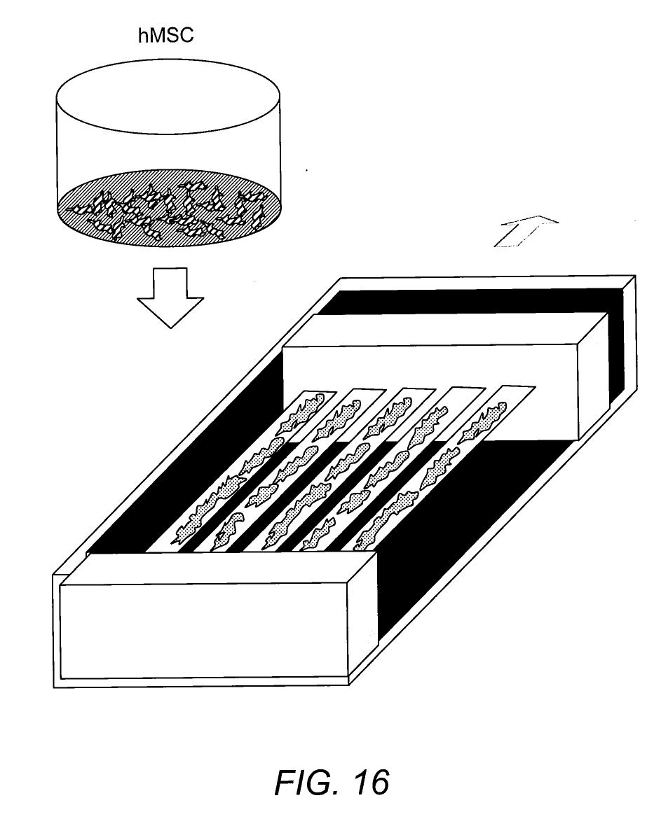

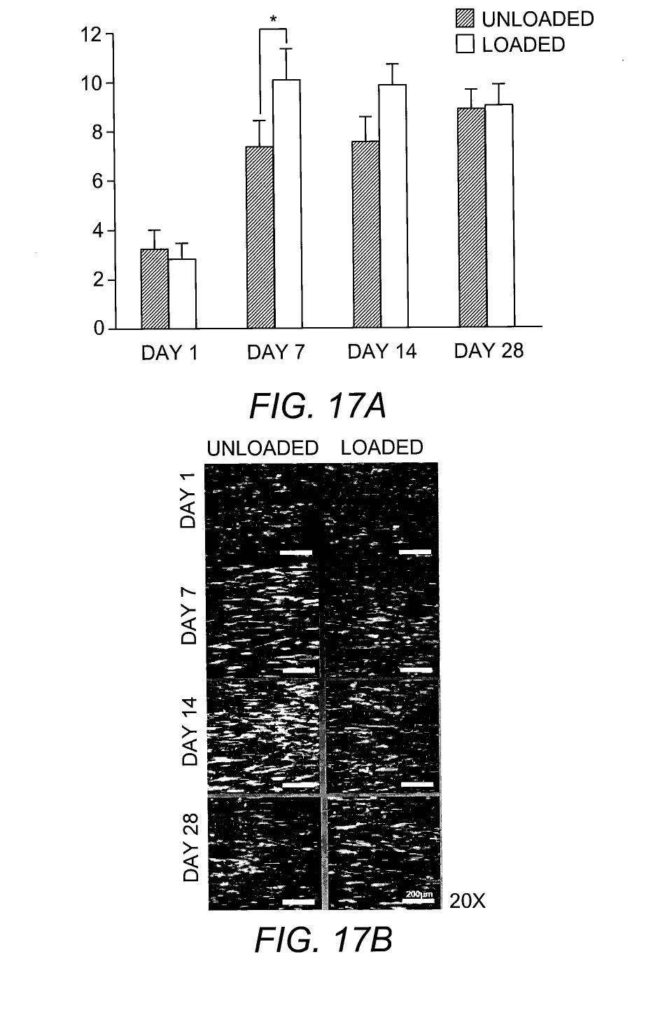

The effect of mechanical stimulation on the fibrogenic aligned nanofiber matrix produced in accordance with the method of this application in further enhancing hMSCs to differentiate to fibroblasts was then examined. A modulator bioreactor system designed to apply tensile strain to scaffolds, such as one described in PCT International Application No. PCT/US2009/06453, filed Dec. 8, 2009, which provides configurable displacement and frequency application, was used. See FIG. 16.

Results of cell proliferation studies following mechanical stimulation are shown in FIG. 17. As shown therein, cells remained viable on aligned polymer nanofiber scaffolds exposed (loaded) and unexposed (unloaded) to mechanical stimulation over time with distinct aligned cell orientation. However, cell proliferation on loaded scaffolds exposed to mechanical stimulation increased significantly compared to unloaded controls.

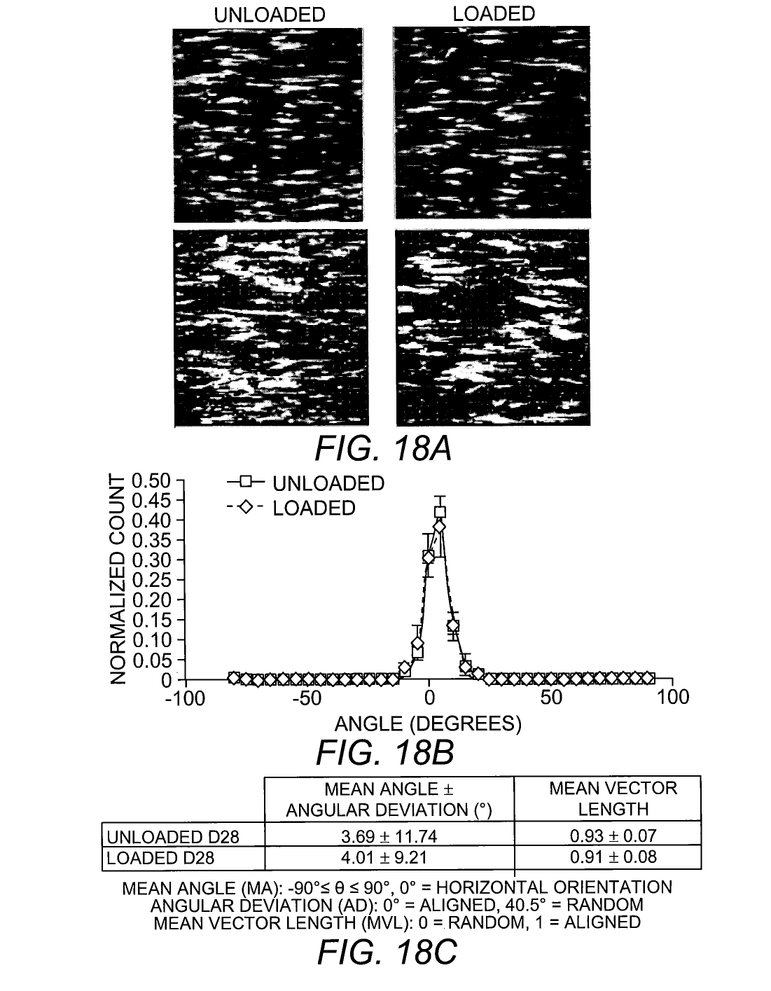

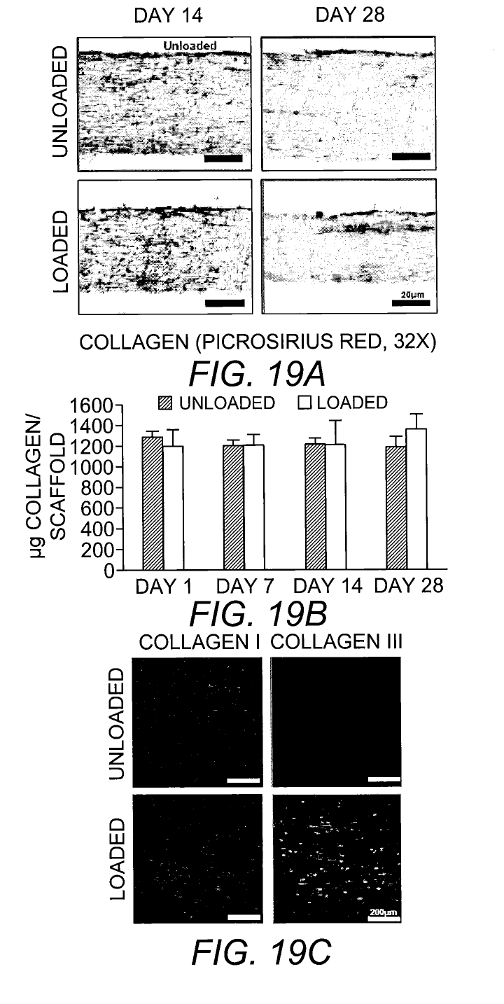

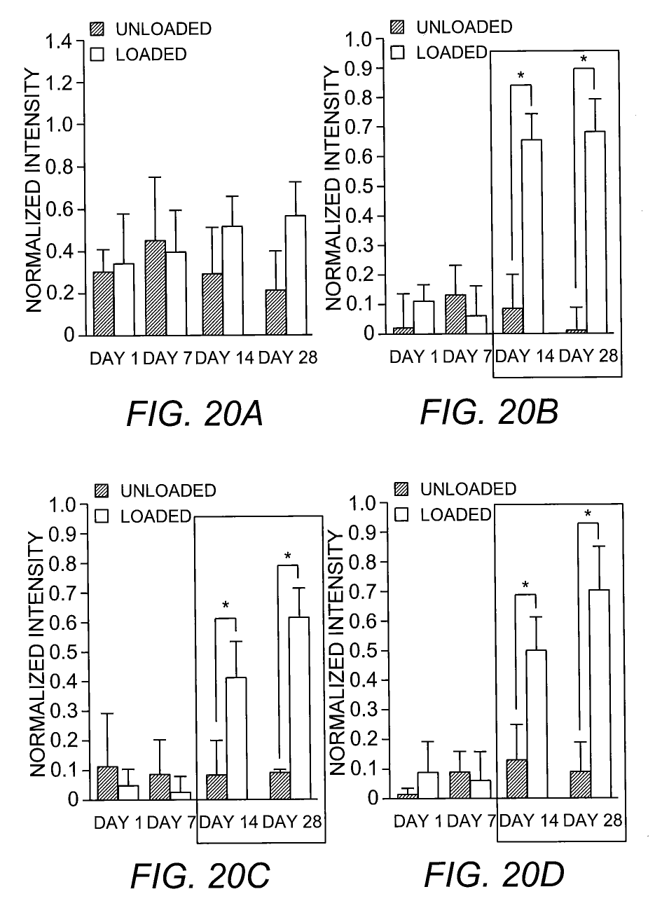

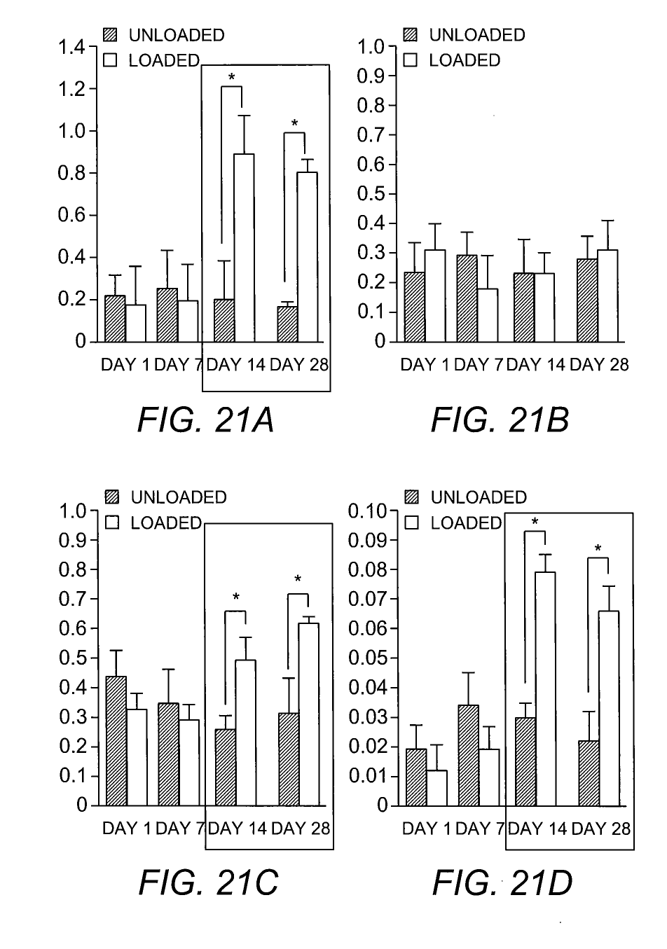

Cell alignment, as shown in FIG. 18, remained similar on both loaded and unloaded scaffolds and no significant difference in total collagen production between groups was observed (see FIG. 19). However, deeper penetration of collagen into loaded scaffolds was observed and was enhanced over time. Further, collagen type II was produced only on loaded scaffolds. In addition, significant up-regulation of collagen III, fibronectin and tenascinC was observed by day 14 in cells of the loaded scaffolds and was maintained after 28 days of culture (see FIG. 20). The up-regulation of fibroblastic markers was coupled with increases in expression of integrin subunits α2, α5 and β1 (see FIG. 21).

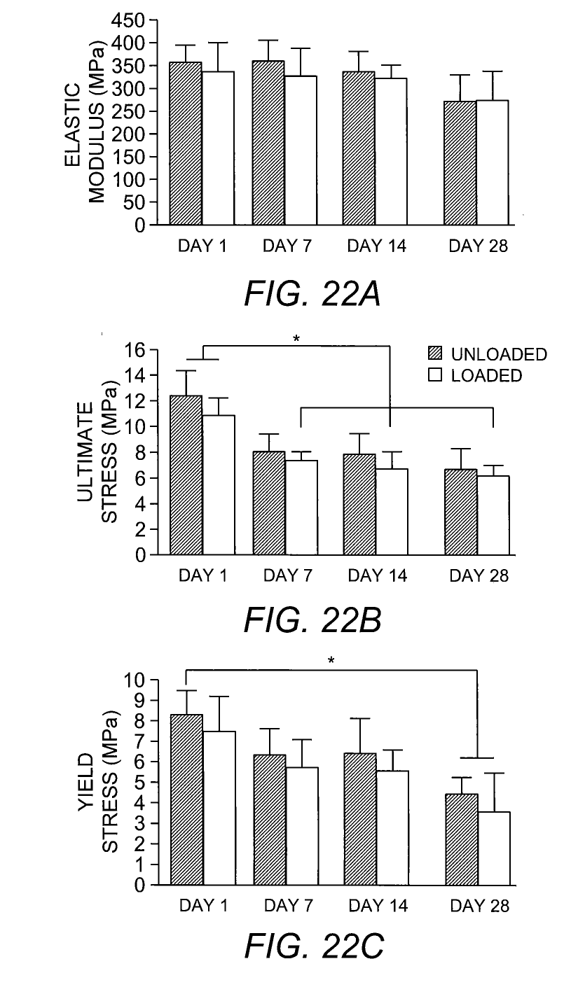

Mechanical properties of the tissue scaffold following mechanical stimulation remained within range of native ACL as scaffolds degraded (see FIG. 22). A significant decrease in ultimate tensile strength (UTS) was observed by day 7 in loaded group and a decrease in yield stress was observed by 28 days of culture in both groups.

While cell alignment was guided predominantly by fiber organization and was not enhanced by mechanical stimulation, nanofiber scaffolds coupled with the mechanical stimulation of tensile loading produced in accordance with the method of this application directed hMSC differentiation towards fibroblast-like phenotype. Further, differentiation was coupled with enhanced cell proliferation and fibroblast-like matrix deposition. The collagen type I:III expression ratio of 8.35±2.12 is indicative of a ligament fibroblast-like phenotype (Amiel et al. J. Orthop. Res. 1984 1(3):257-265) as opposed to a significantly lower type I:III ratio indicative of tendon fibroblasts.

Chemical stimulation via addition of the growth factor bFGF resulted in increased collagen production with tensile loading after 14 days of culture as opposed to 28 days of culture without the growth factor.

In another embodiment, a method is provided for producing tissue scaffolds which direct differentiation of seeded stem cells on the scaffold to chondrocytes.

In this embodiment, the substrate selected comprises a hydrogel and an effective amount of one or more extracellular matrix components.

Non-limiting representative examples of suitable hydrogels for use in this embodiment are composed of a material selected from agarose, carrageenan, polyethylene oxide, polyethylene glycol, tetraethylene glycol, triethylene glycol, trimethylolpropane ethoxylate, pentaerythritol ethoxylate, hyaluronic acid, thiosulfonate polymer derivatives, polyvinylpyrrolidone-polyethylene glycol-agar, collagen, dextran, heparin, hydroxyalkyl cellulose, chondroitin sulfate, dermatan sulfate, heparan sulfate, keratan sulfate, dextran sulfate, pentosan polysulfate, chitosan, alginates, pectins, agars, glucomannans, galactomannans, maltodextrin, amylose, polyalditol, alginate-based gels cross-linked with calcium, polymeric chains of methoxypoly(ethylene glycol)monomethacrylate, chitin, poly(hydroxyalkyl methacrylate), poly(electrolyte complexes), poly(vinylacetate) cross-linked with hydrolysable bonds, water-swellable N-vinyl lactams, carbomer resins, starch graft copolymers, acrylate polymers, polyacrylamides, polyacrylic acid, ester cross-linked polyglucans, and derivatives and combinations thereof.

Nonlimiting examples of suitable extracellular matrix (ECMs) components include proteoglycans such as chondroitin sulfate, aggrecan and/or decorin, collagen type II and collagen type I, as well as combinations thereof. To induce chondrocytes, it may be preferable to select as at least one of the ECM components collagen I.

An ECM-hydrogel scaffold was produced in accordance with the method of this disclosure and shown to induce differentiation of hMSCs into chondrocytes. More specifically, a biomimetic ECM-hydrogel scaffold system for cartilage tissue engineering was designed by incorporating cartilage extracellular matrix (ECM) components, such as chondroitin sulphate (CS) and collagen II in a poly(ethylene glycol) dimethacrylate (PEGDM) hydrogel. This scaffold produced in accordance with the method of this application promoted cell proliferation and chondrogenesis of encapsulated hMSCs while minimizing terminal differentiation into hypertrophic chondrocytes.

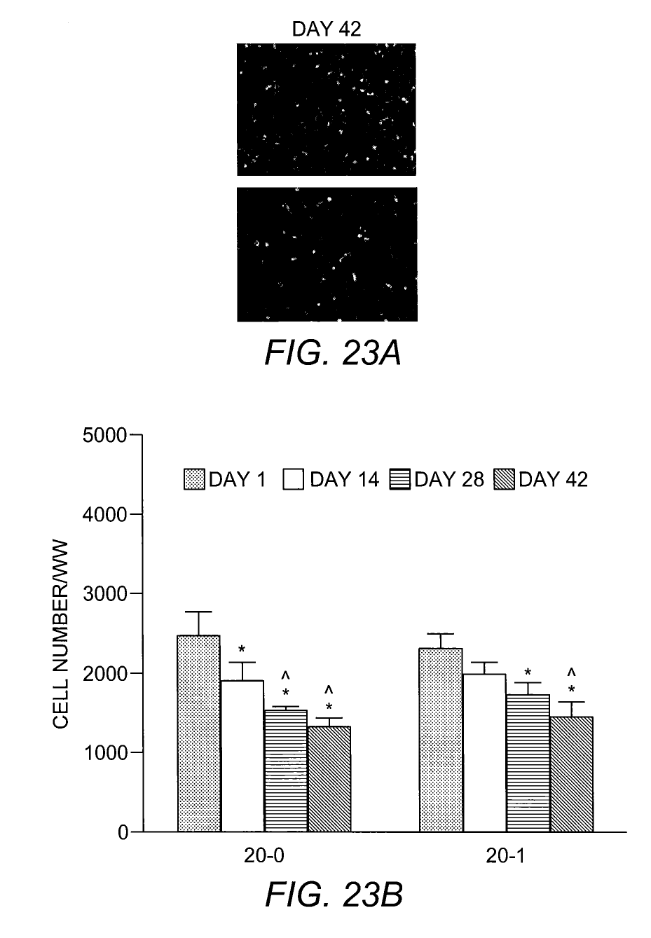

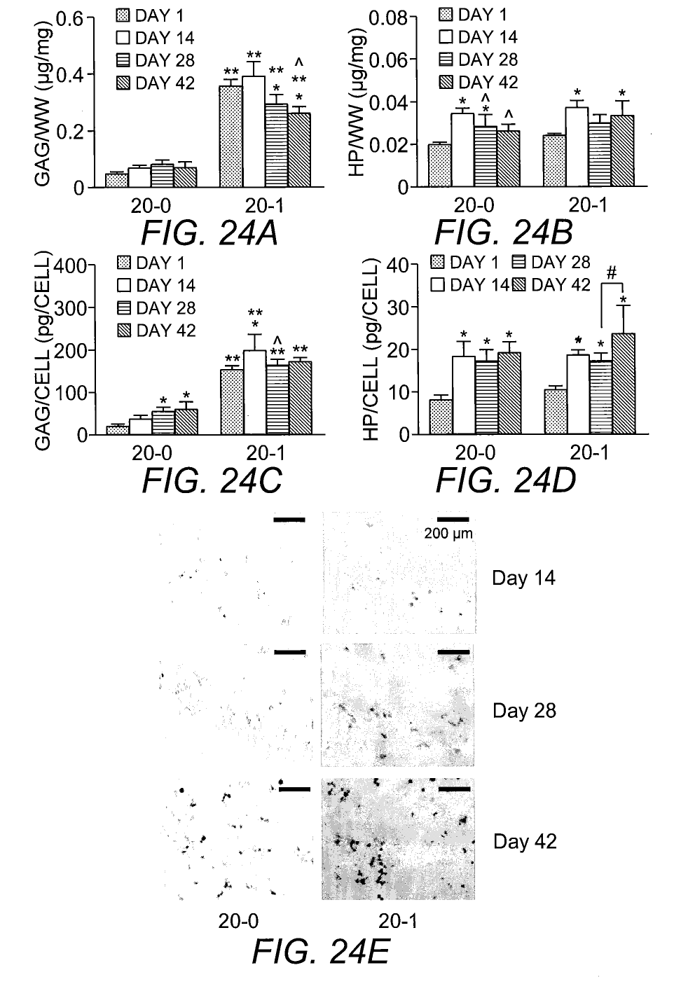

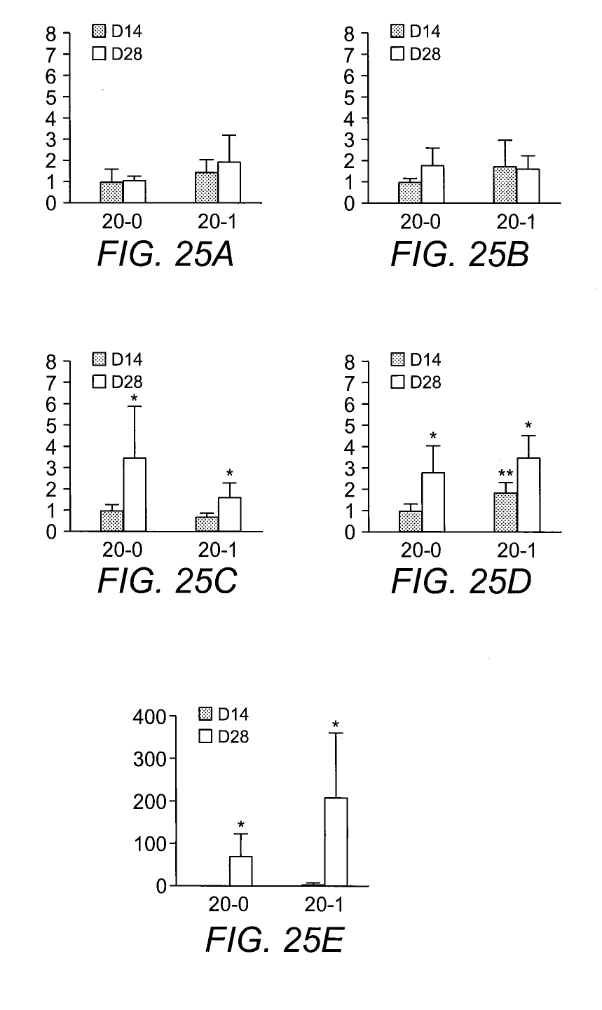

FIGS. 23A and B provide a comparison of cell viability and proliferation with the chondrogenic tissue scaffold (20-1 group) produced in accordance with the method of this application compared to a tissue scaffold without the selected biomaterial/scaffold design parameters directing differentiation to chondrocytes (20-0 group). Cells remained mostly viable over the culturing period for both scaffolds (FIG. 23A) and hMSC cell numbers decreased over time for both scaffolds (FIG. 23B). No significant difference in cell proliferation was observed between the chondrogenic tissue scaffold and the tissue scaffold without the selected biomaterial/scaffold design parameters directing differentiation to chondrocytes. However, deeper staining with Alcian Blue (a dye that bonds to CS), higher initial GAG in the hydrogels containing CS-6-SH that was incorporated into the network using the DMMB assay for sulfated GAG, and increased swelling with incorporated CS-6-SH (particularly at higher concentrations of CS-6-SH) demonstrate that CS-6-SH was incorporated into the PEGDM hydrogel scaffold. In addition, the presence of 1 wt. % CS-6-SH in the chondrogenic tissue scaffold upregulated collagen I gene expression relative to the tissue scaffold without the selected biomaterial/scaffold design parameters directing differentiation to chondrocytes at Day 14 (before administration of chondrogenic media). At Day 28, significant upregulation of COMP and collagen markers (collagen I and II) was noted for both the chondrogenic tissue scaffold and the tissue scaffold without the selected biomaterial/scaffold design parameters directing differentiation to chondrocytes. However, a statistically significant increase was observed for collagen I at Day 14 for the chondrogenic tissue scaffold produced in accordance with the method of this application (see FIG. 25D). While the concentration of CS-6-SH utilized in this study did not result in significant promotion of chondrogenesis with the incorporation of CS-6-SH, it is expected that further optimization of the PEGDM:CS-6-SH hydrogel scaffold at a range between about 10-20 w/v % (between 100 and 200 mg) PEGDM and about 0.1 to 4 w/v % (1 to 40 mg/ml) of CS-6-SH will serve to promote hMSC chondrogenesis as compared to the tissue scaffold without the selected biomaterial/scaffold design parameters directing differentiation to chondrocytes. It is further expected that other ECM components such as, but not limited to, collagen will be effective in a range between about 0.05 and 1 w/v % (0.5 and 10 mg/ml). It is also expected that a higher seeding density will work synergistically with the ECM component to further enhance chondrogenesis of this tissue scaffold as seeding with 15-20 million cell/ml of bovine marrow stem cells has been very effective in enhancing chondrogenesis.

In another embodiment, a method is provided for producing tissue scaffolds which direct differentiation of seeded stem cells on the scaffold to fibrochondrocytes.

In this embodiment, the substrate selected for the tissue scaffold comprises a hydrogel and an effective amount of one or more extracellular matrix components.

Non-limiting representative examples of suitable hydrogels for use in this embodiment are composed of a material selected from agarose, carrageenan, polyethylene oxide, polyethylene glycol, tetraethylene glycol, triethylene glycol, trimethylolpropane ethoxylate, pentaerythritol ethoxylate, hyaluronic acid, thiosulfonate polymer derivatives, polyvinylpyrrolidone-polyethylene glycol-agar, collagen, dextran, heparin, hydroxyalkyl cellulose, chondroitin sulfate, dermatan sulfate, heparan sulfate, keratan sulfate, dextran sulfate, pentosan polysulfate, chitosan, alginates, pectins, agars, glucomannans, galactomannans, maltodextrin, amylose, polyalditol, alginate-based gels cross-linked with calcium, polymeric chains of methoxypoly(ethylene glycol)monomethacrylate, chitin, poly(hydroxyalkyl methacrylate), poly(electrolyte complexes), poly(vinylacetate) cross-linked with hydrolysable bonds, water-swellable N-vinyl lactams, carbomer resins, starch graft copolymers, acrylate polymers, polyacrylamides, polyacrylic acid, ester cross-linked polyglucans, and derivatives and combinations thereof.

Nonlimiting examples of suitable extracellular matrix components (ECMs) include proteoglycans such as chondroitin sulfate, aggrecan and/or decorin, collagen type II and collagen type I, as well as combinations thereof. To induce fibrochondrocytes, it may be preferable to select as ECM components collagen I and II.

In this embodiment, the substrate may further comprise polymer nanofibers and/or microfibers which also induce differentiation of the stem cells to fibrochondrocytes.

Nonlimiting examples of polymers which can be used in the polymeric nanofibers and/or microfibers include biodegradable polymers selected from the group consisting of aliphatic polyesters, poly(amino acids), modified proteins, polydepsipeptides, copoly(ether-esters), polyurethanes, polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, modified polysaccharides, polycarbonates, polytyrosinecarbonates, polyorthocarbonates, poly(trimethylene carbonate), poly(phosphoester)s, polyglycolide, polylactides, polyhydroxybutyrates, polyhydroxyvalerates, polydioxanones, polyalkylene oxalates, polyalkylene succinates, poly(malic acid), poly(maleic anhydride), polyvinylalcohol, polyesteramides, polycyanoacrylates, polyfumarates, poly(ethylene glycol), polyoxaesters containing amine groups, poly(lactide-co-glycolides), poly(lactic acid)s, poly(glycolic acid)s, poly(dioxanone)s, poly(alkylene alkylate)s, biopolymers, collagen, silk, chitosan, alginate, and a blend of two or more of the preceding polymers.

In this embodiment, the architecture of the polymeric nanofibers and/or microfibers can be aligned or unaligned.

In yet another embodiment, a method for producing an osteogenic tissue scaffold is provided.

In one embodiment of this method, the substrate selected comprises a composite of polymer and an effective amount of bioglass (BG) or glass ceramic, the degradation of which produces relevant ions at a concentration and temporal distribution that directs differentiation of stem cells to osteoblasts. The advantages of a polymer-BG composite are that it is bioactive thus forming a Ca—P layer, it neutralizes acidic and basic degradation products, it increases mechanical strength, and it increases structural integrity. Nonlimiting examples of bioglasses useful in this embodiment are described by Hench et al.(Science. 1984 Nov. 9; 226(4675):630-6). In one embodiment the bioglass used is 45S5 BG. However, other bioglass or glass ceramic combinations with, for example, 50% or higher of SiO₂ can be used.

In another embodiment of this method, the substrate selected comprises a polymer and the scaffold is exposed to an osteogenic media derived from an osteogenic tissue scaffold comprising a composite of polymer and an effective amount of bioglass or glass ceramic seeded with stem cells.

Nonlimiting examples of polymers which can be used in these osteogenic tissue scaffolds include biodegradable polymers selected from the group consisting of aliphatic polyesters, poly(amino acids), modified proteins, polydepsipeptides, copoly(ether-esters), polyurethanes, polyalkylenes oxalates, polyamides, poly(iminocarbonates), polyorthoesters, polyoxaesters, polyamidoesters, poly(ε-caprolactone)s, polyanhydrides, polyarylates, polyphosphazenes, polyhydroxyalkanoates, polysaccharides, modified polysaccharides, polycarbonates, polytyrosinecarbonates, polyorthocarbonates, poly(trimethylene carbonate), poly(phosphoester)s, polyglycolide, polylactides, polyhydroxybutyrates, polyhydroxyvalerates, polydioxanones, polyalkylene oxalates, polyalkylene succinates, poly(malic acid), poly(maleic anhydride), polyvinylalcohol, polyesteramides, polycyanoacrylates, polyfumarates, poly(ethylene glycol), polyoxaesters containing amine groups, poly(lactide-co-glycolides), poly(lactic acid)s, poly(glycolic acid)s, poly(dioxanone)s, poly(alkylene alkylate)s, biopolymers, collagen, silk, chitosan, alginate, and a blend of two or more of the preceding polymers.

In these embodiments, the architecture selected may be microspheres, nanofibers and/or microfibers, sheets, hydrogels and combinations thereof.

In some embodiments, this method may further comprise exposing the osteogenic tissue scaffold seeded with stem cells to chemical stimulation such as an osteogenic media. An example of a standard osteogenic media is a tissue culture media comprising 1-150 μg/mL Ascorbic Acid, 3-15 mM β-glycerophosphate, and 0-10⁻⁶M Dexamethasone (Kadiyala et al. Cell Transplant. 1997 6(2):125-34; Jaiswal et al. J Cell Biochem. 1997 64(2):295-312; D'Ippolito et al. Bone. 2002 31(2):269-75; Bruder et al. Clin Orthop Relat Res. 1998 (355 Suppl):S247-56; Fischer et al. Tissue Eng. 2003 9(6):1179-88). Alternatively, as demonstrated herein, the chemical stimulation may comprise a media derived from an osteogenic tissue scaffold comprising a composite of polymer and an effective amount of bioglass or glass ceramic seeded with stem cells. In one embodiment, this osteogenic media is obtained by submerging the osteogenic tissue scaffolds in standard culture media, and then removing the media for use for stimulating stem cells. Many components of the biomaterial including, but not limited to, silicon, calcium, and phosphate ions are believe to play a role in the osteogenesis of stem cells. In another embodiment, this osteogenic media can be prepared by adding critical quantities of these various ions to a standard culture media.

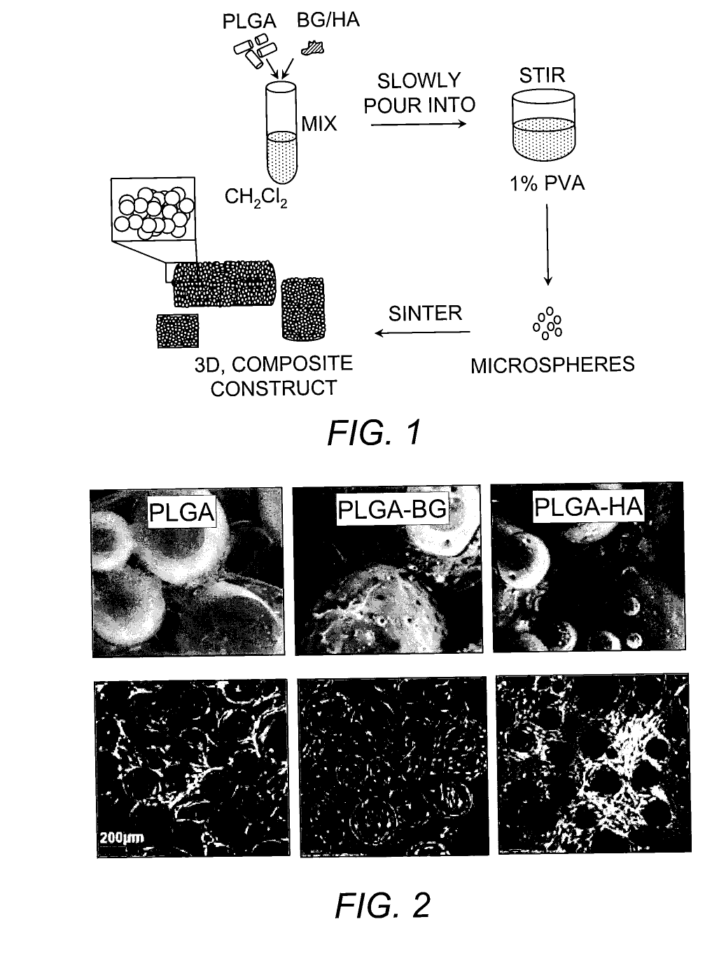

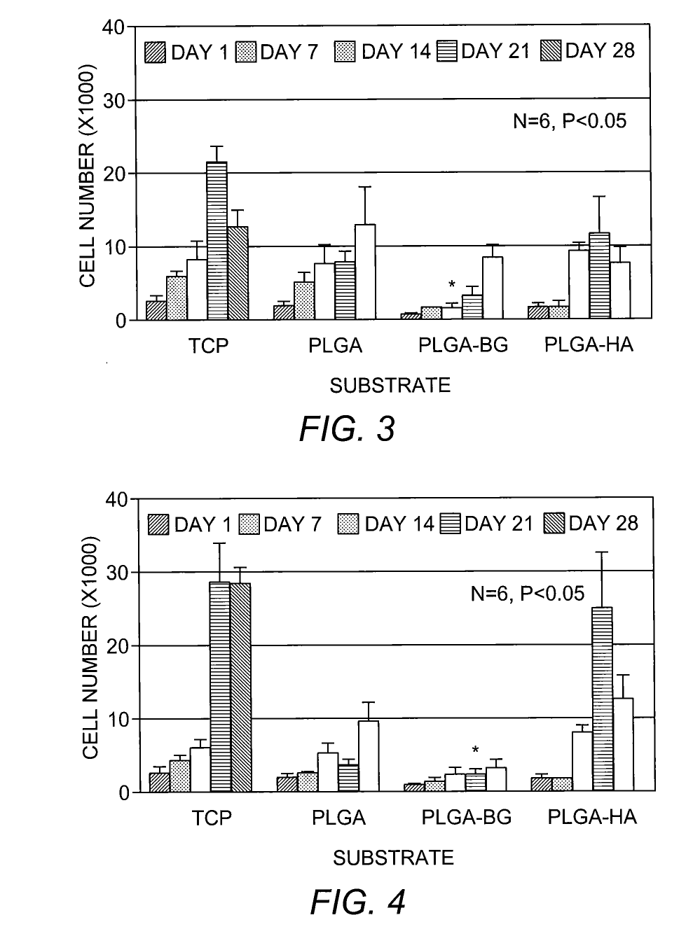

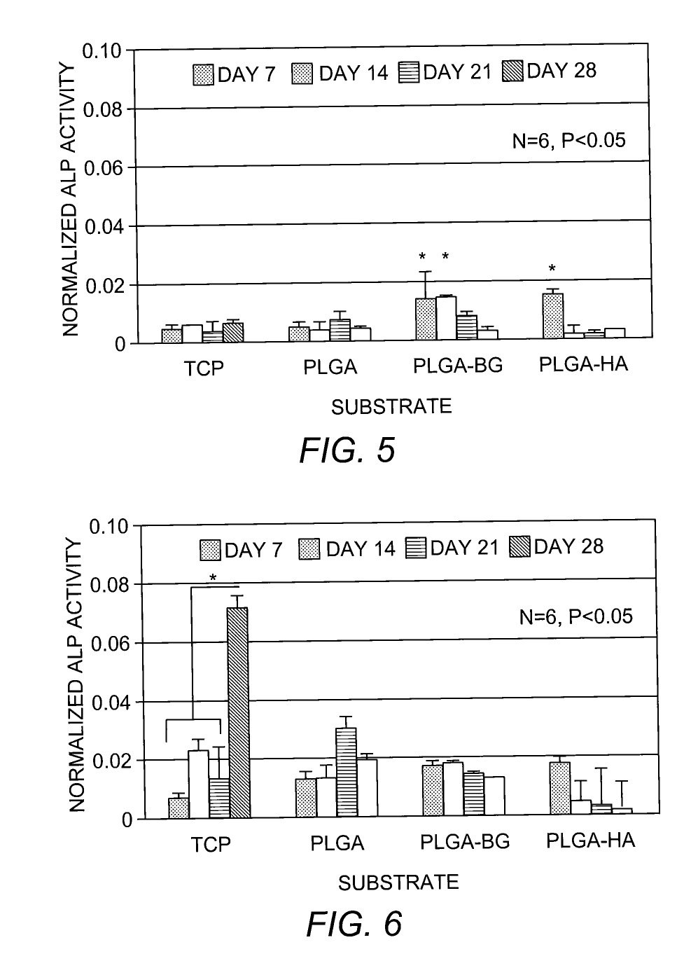

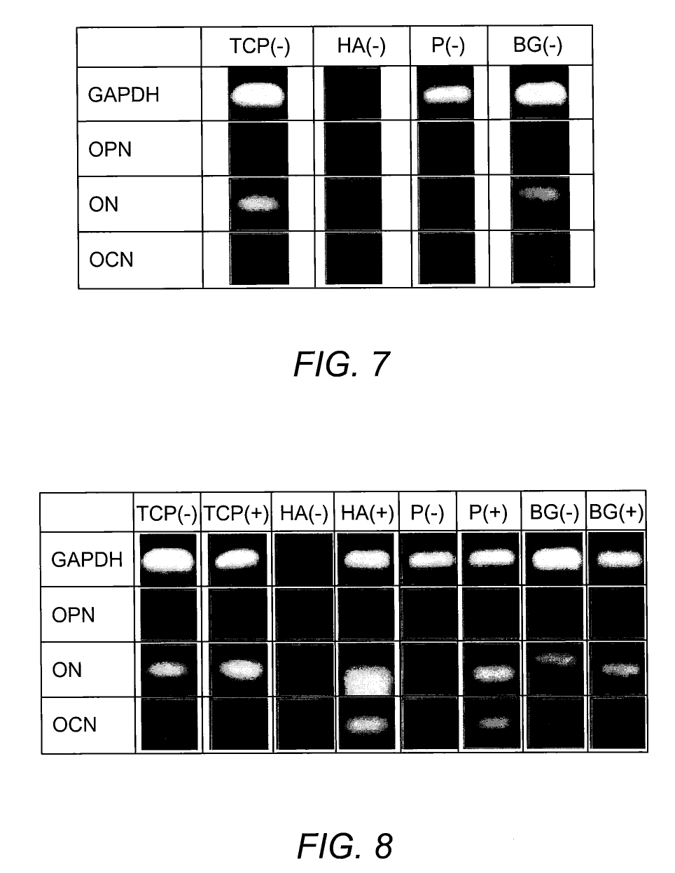

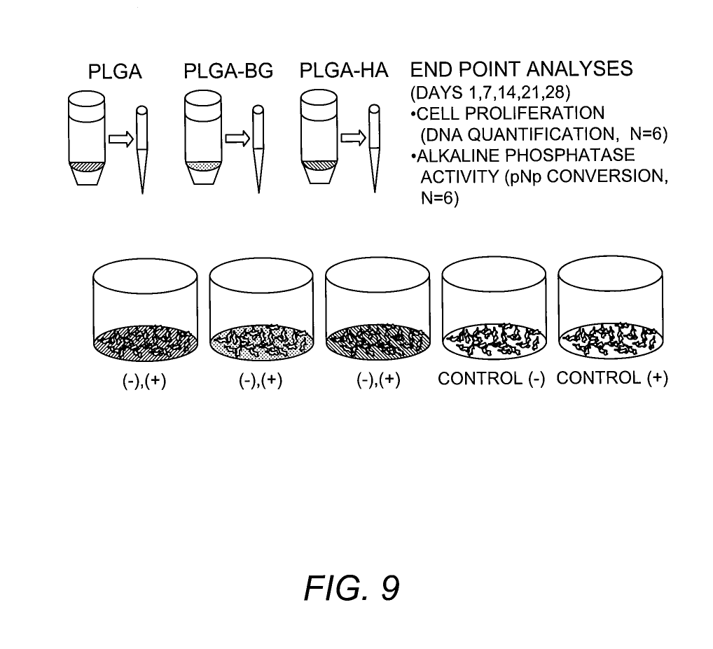

An osteogenic tissue scaffold was produced in accordance with the method of this application and as outlined in FIG. 1. The effect of the selected substrate on cell distribution and viability is shown in FIG. 2. The effect of the selected substrate on cell proliferation is shown in FIG. 3. The effect of the selected substrate and standard osteogenic media on cell proliferation is shown in FIG. 4. The effect of the selected substrate on ALP activity is shown in FIG. 5. The effect of the selected substrate and standard osteogenic media on ALP activity is shown in FIG. 6. The effect of the selected substrate on gene expression is shown in FIG. 7. The effect of the selected substrate and osteogenic media on regulation of bone proteins is shown in FIG. 8.

As demonstrated by the results of FIGS. 2-8, PLGA-BG composite was osteoinductive and promoted the highest level of ALP activity and expression of osteoblastic markers in the absence of osteogenic media. In contrast, PLGA-HA composite supported hMSC differentiation only in presence of standard osteogenic media.

Additional experiments were conducted with hMSCs cultured in media derived from the osteogenic tissue scaffold comprising a composite of polymer and an effective amount of bioglass or glass ceramic seeded with stem cells.

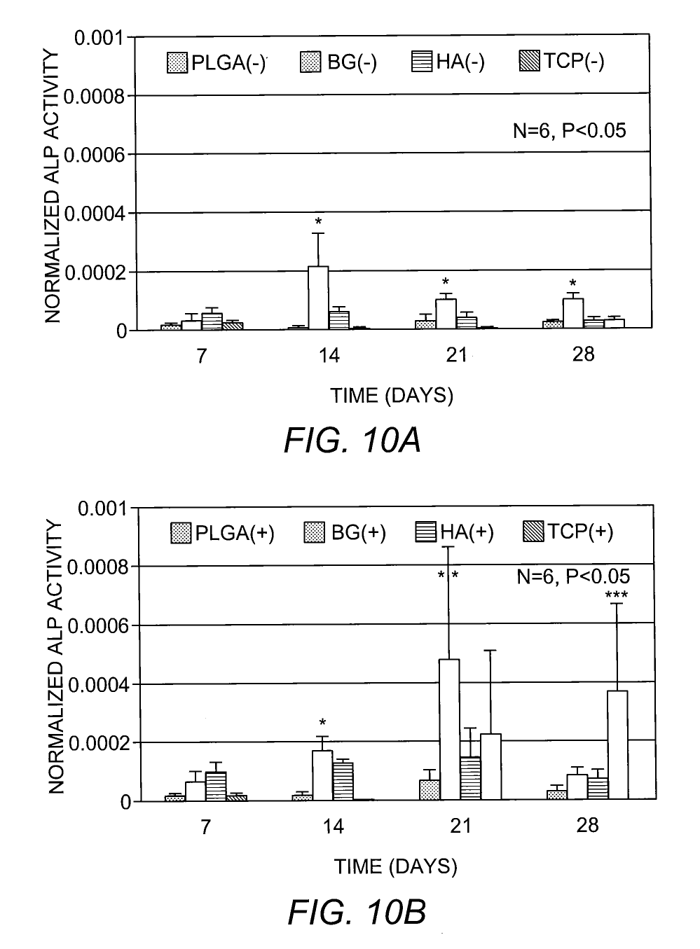

ALP activity graphed against time for hMSCs cultured in this osteogenic media compared to media derived from scaffolds of PLGA, PLGA-HA and TCP are shown in FIG. 10. ALP activity was minimal in hMSCs treated with PLGA and TCP media. Higher ALP activity was found in hMSCs treated with composite-conditioned media. hMSCs treated with PLGA-BG media exhibited significantly higher ALP activity which peaked on day 14. When normalized, ALP activity was higher at day 14, and peaked on day 21 for hMSCs treated with PLGA-BG (+) as compared to PLGA-HA and TCP treated cells.

Tissue scaffolds produced in accordance with the methods disclosed in this application can be used to direct differentiation of seeded stem cells on the tissue scaffold to osteoblasts, fibrochondrocytes, chondrocytes or fibroblasts and thus have a variety of uses in bone, tendon and cartilage repair.

Throughout this application, certain publications are referenced. The disclosures of these publications are hereby incorporated by reference into this application in order to more fully describe the state of the art as of the date of the invention described and claimed herein.

The following section provides further illustration of the methods and apparatuses of the present invention. These examples are illustrative only and are not intended to limit the scope of the invention in any way.

### EXAMPLES

### Example 1

### Materials and Methods for Tissue Scaffolds Directing Osteogenic Differentiation

Three-dimensional tissue constructs were prepared in accordance with the schematic of FIG. 1 and procedure of Lu et al. (J Biomed Mater Res A. 2003 64(3):465-74) from the following 4 substrates:

PLGA Polylactide-co-glycolide 85:15 (Alkermes, Waltham, Mass.)

PLGA-BG 20% 45S5 bioactive glass, (MO-SCI, Rolla, Mo.)

PLGA-HA 20% hydroxyapatite (Plasma Biotal, Tideswell, UK); and

TCP (Tissue culture polystyrene)

The constructs were seeded with human mesenchymal stem cells (Lonza Walkersville Inc., Walkersville, Md.) at 3000 cells/cm².

### Example 2

### Materials and Methods for Osteogenic Differentiation of hMSCs in Bioglass-PLGA Conditioned Media

A schematic of the experimental design is shown in FIG. 12.

PLGA, PLGA-HA, PLGA-BG and TCP scaffolds were submerged in Dulbecco's Modified Eagle's Media (DMEM) with 10% fetal bovine serum, 1% penicillin-streptomycin, and 1% non-essential amino acids. (0.1 mg/mL) for at least 2 days and conditioned media from each of the different scaffolds was obtained.

hMSCs seeded at 3000 cells/cm² on 48-2311 TCP plates were then treated with conditioned media (−) or conditioned media with 50 μg/mL AA, 10 mM 13-GP, 0.1 μM Dexamethasone (+).

End Point Analyses (on days 1, 7, 14, 21 and 28) included cell proliferation (DNA quantification, n=6) and ALP activity (pNp conversion, n=6).

### Example 3

### Materials and Methods for Tissue Scaffolds Directing Fibrogenic Differentiation

Aligned and unaligned PLGA (85:15) nanofiber scaffolds were produced by electrospinning in accordance with the procedure of Moffat et al. (Tiss. Eng. A 2009 15(1):115-26). hMSCs were obtained from Lonza Walkersville Inc. Human rotator cuff fibroblasts (hRCFs) were derived from explant cultures of human rotator cuff tendon tissue. Cells (hMSC and hRCF) were seeded on the nanofiber scaffolds at a density of 3.14×10⁴ cells/cm² and maintained in fully supplemented Dulbecco's Modification of Eagle's Media (Cellgro, Mediatech Inc., Manassas, Va.), with 10% fetal bovine serum (Embryonic Stem Cell Qualified, Atlanta Biologicals, Lawrenceville, Ga.), 1% Penicillin-Streptomycin (Cellgro), 1% Non-essential Amino Acids (Cellgro), 0.1% Gentamicin Sulfate (Cellgro) and 0.1% Amphotericin B (Cellgro) at 37° C. and 5% CO₂.

The following endpoint analyses were performed:

Cell viability (Live/Dead, n=2, at day 1, 7, 13)
Actin staining (n=2, at day 1, 3)
Integrin α2, αV, α5, β1 Expression (n=5, at day 1, 7, 14)

### Example 4

### Mechanical Stimulation of Tissue Scaffolds Directing Fibrogenic Differentiation

Mechanical stimulation was applied via a modulator bioreactor system designed to apply tensile strain to scaffolds, such as described in PCT International Application No. PCT/US2009/06453, filed Dec. 8, 2009, which provides configurable displacement and frequency application.

hMSCs were commercially obtained (Lonza Walkersville Inc.) and evaluated for stem cell markers CD71, CD90 and CD106 via flow cytometry in accordance with procedures described by Pittinger et al. (Science 1999 284(5411):143-7).

Aligned polymer nanofiber scaffolds of Example 3 were pre-cultured with cells for 5 days. The scaffolds were then subjected to 1% tensile strain for 90 minutes twice daily. Unloaded scaffolds not exposed to mechanical stimulation served as controls.

End-point analysis performed after 1, 7, 14 and 28 days included cell attachment and alignment (n=3), cell proliferation (n=5), quantitative (n=5) and qualitative (n=2) collagen deposition, gene expression of fibroblastic markers (n=3), and determination of tensile mechanical properties (n=5).

### Example 5

### Materials and Methods for Tissue Scaffolds Directing Chondrogenic Differentiation

PEGDM (10 kDa) and CS-6-SH were synthesized in accordance with procedures of Lin-Gibson et al. (Biomacromolecules 2004 5:1280), Tae et al. (Biomacromolecules 2007 8:1979); and Cai et al. (Biomaterials 2005 26:6054). For a 20-1 hydrogel containing 20 w/v % PEGDM and 1w/v % CS-6-SH, 200 mg PEGDM was mixed with 10 mg CS-6-SH and sterilized with 0.2 μm syringe filter. Hydrogels were formulated under cytocompatible, photoinitiating conditions as described by Bryant et al. (Exp. Cell Res 2001 268:189).

Commercially obtained hMSCs (Lonza) were encapsulated at a density of 7M cells/ml.

PEGDM (20 wt. %) with 1 wt. % CS-6-SH (20-1) group served as the experimental group and PEGDM (20 wt. %) hydrogels (20-0) served as control.

Samples were treated with ITS-supplemented DMEM with 50 μg/ml ascorbic acid from Days 1-14; and with DMEM supplemented with insulin, human transferrin and selenous acid (ITS-supplemented DMEM) containing 50 μg/ml ascorbic acid, 10 ng/ml TGF-β3 (Invitrogen) and 100 nM Dexamethasone from Days 14-42.

End-point analyses performed after 1, 14, 28 and 42 days of culture included cell viability was evaluated using Live/Dead assay (Invitrogen), cell proliferation (n=6) measured by DNA quantitation (PICOGREEN®, Molecular Probes), collagen synthesis quantified (n=6) with the hydroxyproline assay and visualized (n=2) with Picrosirius Red staining, sGAG synthesis quantified (n=6) with the DMMB assay and visualized (n=2) with Alcian Blue staining, and expression (n=5) of aggrecan, collagen I and II, COMP and SOX9 evaluated by real time RT-PCR (normalized to GAPDH) using SYBR green as fluorescent dye.

ANOVA and Tukey-Kramer post-hoc test were used for all pair-wise comparisons (p<0.05).

The following claims should not be construed as limiting the invention in any way. One of skill in the art will appreciate that numerous modifications, combinations, rearrangements, etc. are possible without exceeding the scope of the claimed invention. While this invention has been described with an emphasis upon various embodiments, it will be understood by those of ordinary skill in the art that variations of the disclosed embodiments can be used, and that it is intended that the invention can be practiced otherwise than as specifically described and claimed herein.

1. A method for producing a tissue scaffold which directs differentiation of seeded stem cells on the scaffold to a selected cell type, said method comprising
(a) selecting a substrate from which the tissue scaffold is produced;
(b) selecting an architecture for the tissue scaffold;
(c) producing a tissue scaffold with the selected architecture from the selected substrate; and
(d) seeding the tissue scaffold with stem cells so that they differentiate into the selected cells.

2. The method of claim 1 further comprising exposing the tissue scaffold to a physical or mechanical and/or chemical stimulation which directs differentiation of the seeded stem cells on the scaffold to the selected cell type.

3. The method of claim 2 wherein the selected cell type to which seeded stem cells are directed to differentiate is osteoblasts, chondrocytes, fibrochondrocytes or fibroblasts.

4. The method of claim 3 wherein the selected cell type is fibroblasts, the substrate selected for the tissue scaffold comprises polymeric nanofibers and/or microfibers and the architecture is aligned nanofibers and/or microfibers.

5. The method of claim 4 wherein the tissue scaffold is exposed to mechanical stimulation.

6. The method of claim 5 wherein the mechanical stimulation is cyclic tensile loading.

7. The method of claim 4 wherein the tissue scaffold is exposed to chemical stimulation.

8. The method of claim 7 wherein the chemical stimulation is a growth factor.

9. The method of claim 3 wherein the selected cell type is chondrocyte and the substrate selected for the tissue scaffold comprises a hydrogel and an effective amount of one or more extracellular matrix components.

10. The method of claim 9 wherein the one or more extracellular matrix components is a proteoglycan, collagen type II or collagen type I.

11. The method of claim 10 wherein the proteoglycan is selected from the group consisting of chondroitin sulfate, aggrecan and decorin.

12. The method of claim 3 wherein the selected cell type is fibrochondrocyte and the substrate selected for the tissue scaffold comprises a hydrogel and an effective amount of one or more extracellular matrix components.

13. The method of claim 12 wherein the one or more extracellular matrix components is a proteoglycan, collagen type II or collagen type I.

14. The method of claim 13 wherein the one or more extracellular matrix components are collagen type II and collagen type I.

15. The method of claim 13 wherein the proteoglycan is selected from the group consisting of chondroitin sulfate, aggrecan and decorin.

16. The method of claim 12 wherein the substrate further comprises polymeric nanofibers and/or microfibers.

17. The method of claim 16 wherein the architecture of the polymeric nanofibers and/or microfibers is aligned.

18. The method of claim 16 wherein the architecture of the polymeric nanofibers and/or microfibers is unaligned.

19. The method of claim 3 wherein the selected cell type is osteoblasts, the substrate selected for the tissue scaffold comprises a composite of polymer and an effective amount of bioglass or glass ceramic, and the architecture of the tissue scaffold is selected from the group consisting of microspheres, nanofibers and/or microfibers, sheets, hydrogels and combinations thereof.

20. The method of claim 19 wherein the tissue scaffold is exposed to an osteogenic media.

21. The method of claim 20 wherein the osteogenic media comprises media derived from an osteogenic tissue scaffold comprising a composite of polymer and an effective amount of bioglass or glass ceramic seeded with stem cells.

22. The method of claim 3 wherein the selected cell type is osteoblasts, the substrate selected for the tissue scaffold comprises a polymer and the tissue scaffold is exposed to an osteogenic media derived from an osteogenic tissue scaffold comprising a composite of polymer and an effective amount of bioglass or glass ceramic seeded with stem cells.

23. A tissue scaffold produced in accordance with a method of claim 1, said tissue scaffold directing differentiation of seeded stem cells on the tissue scaffold to a selected cell type.

24. The tissue scaffold of claim 23 wherein the selected cell type to which seeded stem cells are directed to differentiate is osteoblasts, fibrochondrocytes, chondrocytes or fibroblasts.
